(19) United States
(12) Patent Application Publication
(10) Pub. No.: US 2017/0181807 A1
(43) Pub. Date: Jun. 29, 2017

(54) INJECTABLE MAGNETIC MICROBEADS FOR EN BLOC TISSUE RESECTION

(71) Applicant: Boston Scientific Scimed, Inc., Maple Grove, MN (US)

(72) Inventors: John Hingston, Framingham, MA (US); Alissa Brandon, Cambridge, MA (US); Ellen Kaplan, Irvine, CA (US)

(21) Appl. No.: 15/386,035

(22) Filed: Dec. 21, 2016

Related U.S. Application Data
(60) Provisional application No. 62/271,575, filed on Dec. 28, 2015.

(51) Int. Cl. A61B 34/00; A61K 41/00; A61N 2/02; A61B 17/22; A61B 1/018; A61B 1/00

(52) U.S. Cl. CPC A61B 34/73; A61B 1/018; A61B 1/00137; A61N 2/02; A61B 17/22; A61K 41/0052; A61B 2034/731; A61B 2017/0034

(57) ABSTRACT

This disclosure concerns magnetic or electromagnetic systems and methods for tissue resection. Systems according to the various embodiments of the disclosure include a first component such as a thin plate or a plurality of beads which, in either case, include magnetic materials and are sized to be inserted between mucosal and submucosal tissue layers, and a second component that may be used to apply an attractive (e.g. magnetic) force to the first component.

### CROSS-REFERENCE TO RELATED APPLICATIONS

This application claims the benefit of priority from U.S. Provisional Application No. 62/271,575, filed on Dec. 28, 2015, the entirety of which is incorporated by reference herein.

### FIELD OF THE DISCLOSURE

This application relates to the field of medical devices and medical procedures. More particularly, the application is related to devices and methods for tissue bulking and tissue resection.

### BACKGROUND

Colorectal cancer is among the most common cancers, and is responsible for approximately 60,000 fatalities each year in the United States alone. (Ahmedine Jamal, et al., Cancer Statistics 2002, CA: A Cancer Journal for Clinicians, Volume 52, pp 23-47 (2002)). Early detection and treatment of these cancers is critical for patient survival: five year survival rates are about 90% among those with localized tumors, but are less than 10% for patients with metastases. (Robert A. Smith et al., Colorectal Cancer (in Holland-Frei Cancer Medicine, 6th Ed., Kufe et al., eds. (2003))). The American Cancer Society recommends periodic screening for colon cancer among older adults, but fewer than 30% of older adults have ever been screened. (Id.)

Colonoscopy is a particularly useful colon cancer screening method, which involves the insertion of a specialized endoscope (a “colonoscope”) into the large bowel to permit direct visual inspection of the lower digestive tract and, if necessary, resection of small tumors (also referred to as “polyps”). In addition, colonoscopy is frequently employed if another screening method (such as detection of occult blood in the stool or sigmoidoscopy) suggests that cancer may be present. But polyp resection (“polypectomy”) using colonoscopy requires a high degree of skill and training, and may accordingly be costly and time consuming.

In a typical colonoscopic polyp resection (“polypectomy”) procedure, the endoscopist must dissect the polyp (which may be as small as several millimeters in diameter) away from the healthy intestinal mucosa surrounding it, while minimizing penetration of the underlying submucosal tissue, which may result in damage or perforation of the intestine. To facilitate the procedure, the endoscopist may inject saline beneath the polyp to help separate it from the submucosal tissue, however the saline will dissipate rapidly once cutting begins. Thus, current methods may facilitate the separation of the polyp from the submucosa at the beginning of the resection process, but not at the end. A more durable separation between polyp and submucosa, if achieved, could further simplify polyp resection significantly while reducing the risk of bowl perforation or submucosal damage.

### SUMMARY

The present disclosure, in its various aspects, provides improved systems and methods for tissue resection in which polyps or other mucosal regions to be resected are durably and effectively raised and separated from submucosal tissue layers.

In one aspect, the present disclosure relates to a system for resecting tissue that includes a magnetically susceptible body insertable into a tissue wall and an electromagnet that is connectable to, or insertable through a working channel of, an endoscope. The magnetically susceptible body is sized and shaped to separate a mucosal layer from a submucosal layer (for instance, it has a thickness of between about 100 microns and about 5 mm). The electromagnet is optionally disposed on or in an endcap connectable to the endoscope. In some cases, the magnetically susceptible body is a particle, particularly a polymer particle (or plurality of particles) comprising a ferromagnetic or paramagnetic material. The particle may include one or more of poly(d-lactic-co-glycolic) acid, polymethylmethacrylate, and/or magnetite. In some cases, the electromagnet is configured to (i.e. is capable of) generating a pulsed magnetic field, which pulsed field may cause a particle (as described above) to move or vibrate. The system optionally or additionally includes a cutting instrument, which may be a mechanical (e.g. having a blade) cutting instrument fixedly attached to the electromagnet and spaced therefrom so as to cut through a mucosal tissue layer when the magnetically susceptible body is disposed beneath the mucosal tissue layer and the electromagnet is energized. Alternatively, the cutting instrument may utilize non-magnetic energy (such as laser, radiofrequency, ultrasound, etc.) to achieve resection. In some instances, the electromagnet is insertable through a working channel of the endoscope or attachable to a side of the endoscope (in a so-called “sidecar” position). These systems may be used in medicine, particularly in polypectomy procedures. In some cases, the magnetically susceptible body includes a flat surface (in which case it may be inserted so that its flat surface is generally parallel to a submucosal tissue), and in some instances the magnetically susceptible body is reversibly attached to a catheter or wire, in which case the insertion of the magnetically susceptible component into the wall of the colon includes separating the magnetically susceptible component from the catheter or wire.

In another aspect, the present disclosure relates to a method for resecting a polyp in a colon of a patient that includes inserting a colonoscope into the colon of the patient, inserting a magnetically susceptible body into a wall of the colon between the polyp and an underlying submucosal tissue, positioning an electromagnet proximate to the polyp and energizing the electromagnet, thereby attracting the magnetically susceptible body to the electromagnet and separating the polyp from the submucosal tissue, and resecting the polyp. In instances, as described above, where the magnetically susceptible body includes include a flat surface, it may be inserted so that its flat surface is generally parallel to a submucosal tissue. In instances where the magnetically susceptible body is reversibly attached to a catheter or wire, the insertion of the magnetically susceptible component into the wall of the colon includes separating the magnetically susceptible component from the catheter or wire. In other cases, the magnetically susceptible body is fixedly attached to a catheter or wire, and the method includes retracting the wire and the magnetically susceptible component after the polyp is resected.

In another aspect, the present disclosure relates to a method for resecting a colonic polyp that includes inserting a magnetically susceptible body into a portion of the colon between the mucosal and submucosal tissue and applying a magnetic field to the magnetically susceptible body, thereby separating the polyp with the mucosal tissue away from the submucosal tissue. In some instances, applying the magnetic field to the magnetically susceptible body includes advancing an electromagnet into the colon proximate to the polyp and activating the electromagnet, in which case the electromagnet is optionally attached to a colonoscope. In some cases, the method involves applying a static field to attract the magnetically susceptible body, thereby urging the polyp away from the submucosal tissue. Alternatively or additionally, the method involves applying a pulsed field, thereby moving or vibrating the magnetically susceptible body and physically separating the polyp from the submucosal tissue. The magnetically susceptible body optionally includes a flat surface, which is inserted generally parallel to the submucosal tissue.

In yet another aspect, the present disclosure relates to a method of resecting endothelial tissue which spares underlying tissue. The method utilizes magnetically susceptible bodies as described above, which bodies are inserted into a wall of a body lumen so as to separate a tissue layer near the lumen center from an underlying tissue layer, facilitating the resection and reducing the risk of penetration or damage to the underlying tissue layer. In various embodiments, the body lumen is a portion of the digestive tract, a uterus, a ureter, an esophagus, a blood vessel, or any other suitable body lumen.

### DRAWINGS

Aspects of the disclosure are described below with reference to the following drawings in which like numerals reference like elements, and wherein:

FIGS. 1A and 1B show a schematic view of an exemplary magnetic tissue separation system including an electromagnet disposed on an endoscope. FIGS. 1C and 1D show magnetically susceptible bodies used in exemplary magnetic tissue separation systems.

FIGS. 2A and 2B show a schematic view of an exemplary magnetic tissue separation system which utilizes a plurality of magnetically susceptible particles and/or a magnetic cutting device.

### Unless otherwise provided in the following specification, the drawings are not necessarily to scale, with emphasis being placed on illustration of the principles of the invention.

### DETAILED DESCRIPTION OF THE PREFERRED EMBODIMENTS

In general, the various systems and methods described herein utilize magnetism or electromagnetism to achieve separation of tissue to be resected from the underlying submucosa. Systems of the present disclosure, according to some embodiments, include a first component, such as a solution, bolus of particles, or a single body such as a disc, that may be disposed beneath a mucosal layer and a second component that exerts an attractive force, such as a magnetic force, on the first component, thereby urging the overlying mucosal layer away from the underlying submucosa. The second component is, optionally, connected or connectable to a medical instrument such as a colonoscope and/or capable of being reversibly activated (for instance, an electromagnet that is activated by supplying current and deactivated by stopping current), so that a user may selectively separate mucosal and submucosal tissue. In use, the first component is disposed (e.g. inserted or deposited) into the tissue at a junction between the mucosal and submucosal tissue layers, then the second component is brought into proximity with the portion of the mucosa disposed over the first component (and optionally activated, if it is configured to do so), thereby urging the mucosal tissue to be resected toward the second component, separating it from the underlying submucosa.

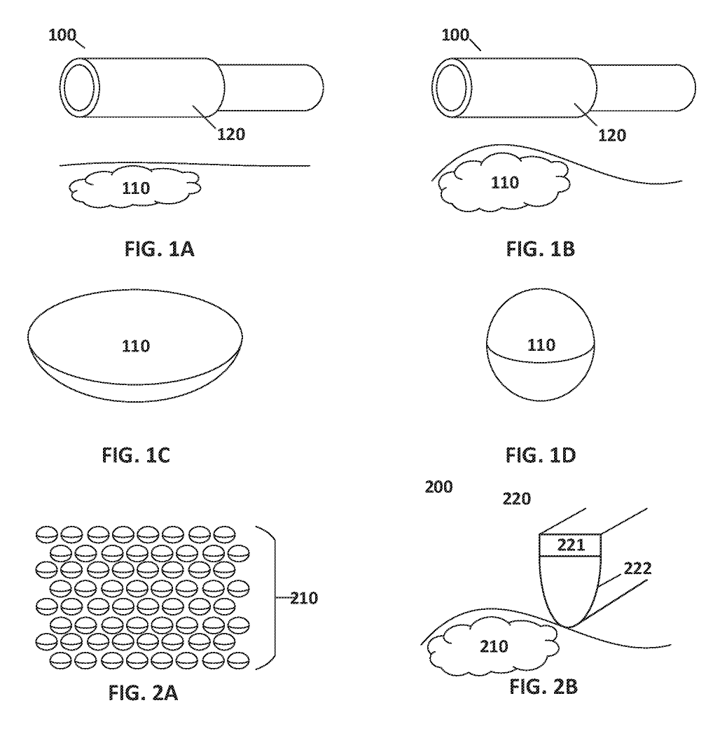

Turning to FIGS. 1A-1D, in some cases, a polypectomy system 100 includes a first component 110 that includes a material that is responsive to a magnetic force (e.g. a paramagnetic or ferromagnetic material). The first component 110 may be a thin plate or disc, a ball, or any other suitable shape, but it is preferably sized to be inserted into a wall of a body lumen such as the colon and to separate an overlying mucosal layer from the underlying submucosa. The system 100 also includes a second component 120, which is preferably an electromagnet configured to deliver a varying magnetic field. The second component 120 may be disposed on a guidewire or other catheter- or wire-platform, or it may be attached to a colonoscope by means of an end cap or “sidecar” clip. In use, a user inserts a colonoscope into the body of the patient and, once a polyp or other structure to be resected is identified, the first component 110 is inserted into the wall of the colon beneath the structure. The first component 110 may be inserted using a catheter or wire which is extended through a working channel of the colonoscope, and may optionally be detached from such catheter or wire. The second component 120 is then advanced through the colon until it is proximate to (e.g. directly adjacent) the structure, and is then activated, urging the first component 110 toward the second component 120 and thereby separating the structure from the underlying submucosal tissue. The structure is then resected while the second component 120 remains activated. Once resection is complete, the second component is deactivated and, if the first component 110 remained tethered, it is retracted.

FIG. 2A illustrates a related embodiment, in which the first component 210 is a bead or plurality of beads or particles that may form a bolus and that may be acted upon by a magnetic field. The beads or particles are preferably formed from a biocompatible polymer and a ferromagnetic or paramagnetic material, as described in U.S. Pat. No. 7,976,823 by Lanphere et al., which is incorporated by reference herein for all purposes. Specifically, the polymer may be one or more of polyvinyl alcohols, polyacrylic acids, polymethacrylic acids, poly vinyl sulfonates, carboxymethyl celluloses, hydroxyethyl celluloses, substituted celluloses, polyacrylamides, polyethylene glycols, polyamides, polyureas, polyurethanes, polyesters, polyethers, polystyrenes, polysaccharides, polylactic acids, polyethylenes, polymethylmethacrylates, polycaprolactones, polyglycolic acids, poly(lactic-co-glycolic) acids (e.g., poly(d-lactic-co-glycolic) acids), and copolymers or mixtures thereof. In some embodiments, the polymeric matrix may be substantially formed of a highly water insoluble, high molecular weight polymer. An example of such a polymer is a high molecular weight polyvinyl alcohol (PVA) that has been acetalized. The polymeric matrix may be substantially pure intrachain 1,3-acetalized PVA and substantially free of animal derived residue such as collagen. In some embodiments, the particle includes a minor amount (e.g., about 2.5 weight percent or less, about one weight percent or less, about 0.2 weight percent or less) of a gelling material (e.g., a polysaccharide, such as alginate). In certain embodiments, the majority (e.g., at least about 75 weight percent, at least about 90 weight percent, at least about 95 weight percent) of the polymeric matrix is formed of a bioabsorbable polymer (e.g., polysaccharide, such as alginate).

The polymer may also be, for example, gel (uncrosslinked) polymer or crosslinked polymer. In some embodiments, the polymer may include one or more of a cross-linked PVA and/or an alginate (e.g., sodium alginate) or other polymeric gel.

The beads or particles also include a ferromagnetic or paramagnetic material (a material that has a magnetic susceptibility of at least about 0.075 or more), for example, a metal (e.g., a transition metal such as nickel, cobalt, or iron), a metal alloy (e.g., a nickel-iron alloy such as Mu-metal), a metal oxide (e.g., an iron oxide such as magnetite), a ceramic nanomaterial, a soft ferrite (e.g., nickel-zinc-iron), a magnet alloy (e.g., a rare earth magnet alloy such as a neodymium-iron-boron alloy or a samarium-cobalt alloy), an amorphous alloy (e.g., iron-silicon-boron), a non-earth alloy, or a silicon alloy (e.g., an iron-zirconium-copper-boron-silicon alloy, an iron-zirconium-copper-boron-silicon alloy). Magnetite is commercially available from FerroTec Corporation (Nashua, N.H.), under the tradename EMG 1111 Ferrofluid. Iron-copper-niobium-boron-silicon alloys are commercially available from Hitachi Metals of America under the tradename Finemet™. Iron-zirconium-copper-boron-silicon alloys are commercially available from MAGNETEC GmbH under the tradename Nanoperm®. In certain embodiments, the ferromagnetic material is a biocompatible material (e.g., magnetite). In some embodiments, the ferromagnetic material is a bioerodible material, such that the material may eventually break down in the body and either be dispersed throughout the body or excreted from the body.

The use of the system 200 according to the embodiments illustrated by FIG. 2B is similar to what has been described above, though the beads or particles 210 are injected into the mucosa beneath the structure to be resected. Once resection is complete, the particles or beads will travel through the lower digestive tract and will be excreted, or they may degrade or resorb.

When beads or particles 210 are used in resection procedures according to certain embodiments of the disclosure, it may be useful to apply a varying (e.g. a pulsed) magnetic field that causes the particles to move or vibrate, thereby improving separation of the overlying structure to be resected from the underlying mucosa.

In some cases, beads or particles 210 are used in conjunction with a specialized magnetic cutting instrument 220, which includes a magnetic or electromagnetic portion 221 and a cutting edge 222 that, together, form a lumen. In use, the cutting instrument 220 is advanced toward a mucosal tissue that has been bulked with particles 210, such that the magnetic or electromagnetic portion 221 exerts an attractive force upon the particles 210, urging them toward the electromagnetic portion and separating the overlying mucosal tissue from the underlying submucosa. The cutting instrument 220 is advanced, bringing the cutting edge 222 into contact with the mucosal tissue and resecting the bulked portion of the tissue as it extends into the lumen.

More generally, the magnetically susceptible materials described herein may be used with other cutting instruments, including, but not limited to, rigid needles or blades, hot or cold cutting loops, biopsy jaws, etc. In some cases, the polyp or other mucosal tissue overlying the magnetically susceptible body is banded (i.e. one or more elastic bands is placed over the tissue).

It should be appreciated that the systems and methods of the present disclosure may be adapted to be used in conjunction with magnetic endoscopic imaging: for instance, the second component may be a magnetic imaging coil disposed on a colonoscope. It should also be appreciated that, although the foregoing examples have focused on resection of colonic polyps, the systems and methods of the present disclosure may be adapted for endoscopic resection of other tissue or tumors, where it is desired to raise and separate a layer to be resected from an underlying layer that is not desired to be resected, such as lesion of the esophagus, upper GI tract, stomach, bladder wall, uterine fibroids, etc.

The phrase “and/or,” as used herein should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” may refer, in one embodiment, to A without B (optionally including elements other than B); in another embodiment, to B without A (optionally including elements other than A); in yet another embodiment, to both A and B (optionally including other elements); etc.

The term “consists essentially of” means excluding other materials that contribute to function, unless otherwise defined herein. Nonetheless, such other materials may be present, collectively or individually, in trace amounts.

As used in this specification, the term “substantially” or “approximately” means plus or minus 10% (e.g., by weight or by volume), and in some embodiments, plus or minus 5%. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

Certain embodiments of the present disclosure have described above. It is, however, expressly noted that the present disclosure is not limited to those embodiments, but rather the intention is that additions and modifications to what was expressly described herein are also included within the scope of the disclosure. Moreover, it is to be understood that the features of the various embodiments described herein were not mutually exclusive and may exist in various combinations and permutations, even if such combinations or permutations were not made express herein, without departing from the spirit and scope of the disclosure. In fact, variations, modifications, and other implementations of what was described herein will occur to those of ordinary skill in the art without departing from the spirit and the scope of the disclosure. As such, the disclosure is not to be defined only by the preceding illustrative description.

1. A system for resecting tissue, comprising:
a magnetically susceptible body insertable into a tissue wall, the material being sized and shaped to separate a mucosal layer from a submucosal layer; and
an electromagnet connectable to, or insertable through a working channel of, an endoscope.

2. The system according to claim 1, wherein the electromagnet is disposed on or in an end-cap connectable to the endoscope.

3. The system according to claim 1, wherein the magnetically susceptible body is a polymer particle comprising a ferromagnetic or paramagnetic material.

4. The system according to claim 3, wherein the polymer particle includes poly(d-lactic-co-glycolic) acid.

5. The system according to claim 3, wherein the polymer particle includes polymethylmethacrylate.

6. The system according to claim 3, wherein the particle incudes magnetite.

7. The system according to claim 3, wherein the electromagnet is configured to create a pulsed magnetic field in response to a user input.

8. The system according to claim 7, wherein the pulsed magnetic field causes the particle to move or vibrate.

9. The system according to claim 1, further comprising a cutting instrument.

10. A method for resecting a polyp in a colon of a patient, comprising the steps of:
inserting, into the colon of the patient, a colonoscope;
positioning a magnetically susceptible body into a wall of the colon between the polyp and an underlying submucosal tissue;
positioning an electromagnet proximate to the polyp;
energizing the electromagnet, thereby attracting the magnetically susceptible body to the electromagnet and separating the polyp from the submucosal tissue; and
resecting the polyp.

11. The method of claim 10, wherein the magnetically susceptible body has a flat surface, and is inserted so that the flat surface is generally parallel to a submucosal tissue.

12. The method of claim 10, wherein the magnetically susceptible body is reversibly attached to a catheter or wire, and the step of inserting the magnetically susceptible component into the wall of the colon includes separating the magnetically susceptible component from the catheter or wire.

13. The method of claim 10, wherein the magnetically susceptible body is fixedly attached to a wire, and the method includes the step of retracting the wire and the magnetically susceptible component after the polyp is resected.

14. A method for resecting a colonic polyp comprising the steps of:
inserting a magnetically susceptible body into a portion of the colon between the polyp and a submucosal tissue; and
applying a magnetic field to the magnetically susceptible body, thereby separating the polyp away from the submucosal tissue.

15. The method of claim 14, wherein the step of applying the magnetic field to the magnetically susceptible body includes advancing an electromagnet into the colon proximate to the polyp and activating the electromagnet.

16. The method of claim 15, wherein the electromagnet is attached to a colonoscope.

17. The method of claim 14, wherein the step of applying the magnetic field includes applying a static field to attract the magnetically susceptible body, thereby urging the polyp away from the submucosal tissue.

18. The method of claim 17, wherein the magnetically susceptible body has a flat surface, and is inserted so that the flat surface is generally parallel to a submucosal tissue.

19. The method of claim 14, wherein the step of applying the magnetic field includes applying a pulsed field, thereby moving or vibrating the magnetically susceptible body and physically separating the polyp from the submucosal tissue.

20. The method of claim 19, wherein the magnetically susceptible body includes a plurality of particles comprising a paramagnetic or ferromagnetic material.
